(19) United States
(12) Patent Application Publication
(10) Pub. No.: US 2013/0345182 A1
(43) Pub. Date: Dec. 26, 2013

(54) STABLE COMPOSITION COMPRISING CHOLESTEROL-LOWERING AGENTS, ANTIHYPERTENSIVE AGENTS AND ANTIPLATLET AGENTS

(71) Applicants: Paola Yazmin Ollervides Rubio, Mexico City (MX); Casimiro Frausto Campos, Mexico City (MX); Antonio Santiago Regalado, Mexico City (MX)

(72) Inventors: Paola Yazmin Ollervides Rubio, Mexico City (MX); Casimiro Frausto Campos, Mexico City (MX); Antonio Santiago Regalado, Mexico City (MX)

(73) Assignee: Silanes Laboratories

(21) Appl. No.: 13/995,936

(22) PCT Filed: Dec. 21, 2010
(86) PCT No.: PCT/MX10/00158

§ 371 Date: Sep. 3, 2013

(51) Int. Cl. A61K 31/616; A61K 31/4178; A61K 31/215

(52) U.S. Cl. CPC A61K 31/616; A61K 31/215; A61K 31/4178
USPC 514/161

(57) ABSTRACT

The present invention relates to a stable composition for providing substances with different types of activity, such as cholesterol lowering agents, antihypertensive agents and antiplatelet agents, which substantially reduce the dosage required to prevent cardiovascular events.

### FIELD OF THE INVENTION

The present invention relates to a stable composition which immediately delivers various drugs having different types of activity such as cholesterol-lowering agents, antihypertensive agents and antiplatelet agents that are useful to control factors that cause major cardiovascular events by preventing and reducing risk for cardiovascular events exhibited by people with high levels of cholesterol and hypertension.

### BACKGROUND OF THE INVENTION

Coronary diseases associated to arterial hypertension and hypercholesterolemia by the Metabolic Syndrome, are among the primary causes of mortality and morbidity.

Coronary heart disease or coronary artery disease (CAD) is characterized by the accumulation of fatty deposits along the innermost layer of coronary arteries. Fat deposits may develop in childhood and keep growing and thickening throughout life. This thickening, named atherosclerosis, narrows the arteries and can decrease or block blood flow to the heart.

Risk factors for coronary heart disease are:
High levels of triglycerides and cholesterol
High blood pressure (hypertension)
Physical inactivity
Smoking
Obesity
Diet which is high in saturated fat
Diabetes

Mortality due to coronary artery disease (angina or heart attack) has decreased by 36% in non-diabetic men and by 27% in non-diabetic women. However, the decrease in cardiovascular mortality in male patients suffering from diabetes has not been significant and in diabetic women rather than diminishing, it has increased.

Epidemiological studies, such as the Framingham Heart Study, revealed the impact of diabetes on cardiovascular disease, this 30-year follow-up study showed an increase in prevalence of macrovascular diseases in patients with diabetes, compared with non-diabetic subjects, this increase occurred in both sexes, but is even greater in women with diabetes than in women without diabetes.

Studies such as the Framingham Heart Study, the Seven Countries Study or the MRFIT Study have demonstrated the role of hypercholesterolemia as a major risk factor in episodes of morbidity and mortality of cardiovascular origin. By using studies such as the Lipid Research Clinics Coronary Primary Prevention Trial and the Helsinki Heart Study, it was demonstrated that cholesterol reduction prevents the occurrence of cardiovascular events. This led to the creation of the National Cholesterol Education Program (NCEP) in 1987 in the United States and to its recommendations on the treatment of hypercholesterolemia. Angiographic studies were initiated to anatomically corroborate the findings of these studies, and studies using statins were initiated in accordance to the recommendations from the NCEP. It has been found that statins possess various pharmacological actions. There are four mechanisms by which statins might prevent cardiovascular diseases:
1. Improvement of the endothelial function
2. Modulation of inflammatory response
3. Stabilization of atherosclerotic plaque
4. Prevention of thrombus formation

Other substances used to prevent cardiovascular events refer to the use of antiplatelet agents such as acetylsalicylic acid and the use of various antihypertensive agents.

It is also well known that one of the failures in the effectiveness of treatments is the lack or problems of compliance due to the amount of tablets that individuals with risk factors must include in their daily intake, in addition to the corresponding cost associated with various medicaments, which has led to suggest the development and use of drug combinations, as shown in the following patent documents:
International patent application WO99/011260 proposes combinations of lipid-lowering substance such as statin (atorvastatin) with antihypertensive agents such as a calcium channel blocker, an ACE inhibitor, an angiotensin-II antagonist, a diuretic, an alpha or beta-adrenergic receptor blocking agent or a vasodilator;
International patent application WO99/011263 and U.S. patent application 60/057,275 relate to the use of statins specifically with a calcium channel blocker such as amlodipine;
Mexican application PA/a/2005/013696 proposes a synergistic combination consisting of lipid-lowering substances like simvastatin in combination with hypotensive substances which are antagonists of angiotensin II receptors, such as losartan;
Applications WO 2008/069612 and WO 2008/044862 propose a group of combinations based on the use of a calcium channel blocker such as amlodipine, with an antagonist of angiotensin II receptors, such as losartan;
Application WO 2007073131 relates to a composition containing a hypotensive substance which is an antagonist of angiotensin II receptor in combination with an antiplatelet agent, specifically losartan and acetylsalicylic acid;
U.S. Pat. No. 6,235,311 describes a formulation containing antiplatelet agents in combination with statins such as pravastatin, lovastatin, simvastatin, atorvastatin, or fluvastatin, cervastatin, but stipulates that these combinations have the disadvantage that they are not stable because the statins are degraded in the presence of antiplatelet agents such as acetylsalicylic acid.

In 2003 the concept of the “polypill” was developed. In the article by Stephen Lim et al “Prevention of cardiovascular disease in high-risk individuals in low and middle income countries: effects on health and costs.” The Lancet, Volume 370, No. 9604, pages 2054 to 2062, the authors mention that the staggered introduction of a ‘polypill’ for high-risk cardiac patients in under-developed zones of the world would save 18 million lives in a decade. The authors state that said polypill should contain in a single tablet three key drugs of cardiovascular therapy: a statin, aspirin, and one or more antihypertensive agents. The World Health Organization (WHO) has expressed interest in the development of such products.

The aim of the polypill is to improve secondary prevention, with its benefit centered on individuals at high risk of suffering stroke or coronary event, especially in patients who have had a previous event, diabetics or individuals with high levels of cholesterol and triglycerides. Also, the goal is to reduce the therapy to the intake of only one tablet daily, thus preventing patients from ceasing treatment and be exposed to a cardiovascular event, as well as to reduce the cost of therapy and in this manner make it accessible to a larger number of patients.

However, it is particularly difficult to formulate combinations of three biologically active agents due to differences in inherent physicochemical properties. The present invention consists of a composition with proven stability, in a dosage unit, which contains in a single phase three drugs to be released immediately.

In the prior art only U.S. publication 2008287403 was found, which proposes the use of an inhibitor of the renin-angiotensin system along with an antihypertensive, of a cholesterol lowering agent, of a diuretic or aspirin for the prevention of cardiovascular events. However, this document only discloses, as examples of application, the clinical evaluation of a compound inhibitor of angiotensin converting enzyme (ACE), specifically ramipril.

The stability of a drug is defined as its ability to stay within the established quality specifications during its lifetime in the package that contains it.

Chemical interactions frequently occur between two or more components of the medicaments in the same dosage form, either between the drugs and/or between the drugs and the pharmaceutical excipients. It is also known that small variations in purity, content or proportions of the drug or other ingredients or in the manufacturing process can significantly alter the effect of the final product.

In order to avoid degradation of statin in the presence of acetylsalicylic acid, U.S. Pat. No. 6,235,311 suggests different embodiments that seek to avoid contact between the drugs. For example, it describes the formation of a bilayer tablet, wherein the first layer consists of aspirin in the form of enteric-coated granules, a diluent and a lubricant, while the second layer contains the statin (pravastatin, lovastatin, simvastatin, fluvastatin, atorvastatin or cerivastatin) together with a buffering agent, but said buffering agent is intended solely to reduce gastrointestinal side effects. Another embodiment is that the layer containing the enteric-coated aspirin is in the core of the tablet and the statin is in the outer coating.

Another disclosed embodiment of U.S. Pat. No. 6,235,311 and its corresponding Mexican patent MX218975 is the use of aspirin and/or statin granules coated with one or two layers in order to control the release of aspirin and protect the integrity of the statin, however, said documents do not disclose information that proves that the stability of this composition is achieved. Another alternative indicates that the mixture of aspirin and statin should be encapsulated separately, which is a very complicated process to perform.

This patent also intends to use uncoated acetylsalicylic acid and to mix it with statin granules or powder, however, according to experiments conducted by the applicant of the present invention using different formulations of both drugs with excipients normally used widely in the pharmaceutical industry for the manufacturing of tablets, it was shown that the mixture is not stable over time, so that the formulation of stable compositions requires a solution not found yet.

The problems which are mentioned in the proposals of U.S. Pat. No. 6,235,311 can be confirmed by the fact that Bristol-Myers Company, owner of the patent, only managed to sell a product based on aspirin and statin in the form of a combo, in which the drugs are in separate tablets of pravastatin and aspirin, each placed in its corresponding cavity in the same packaging material or blister.

Therefore, one of the objects of the present invention is to achieve the stabilization of combinations of two or more drugs in ranges that enable the prevention of cardiovascular events in the population at risk, in one or two oral administrations per day, with the tablet containing the biologically active drugs, a cholesterol lowering agent, an antihypertensive agent and an antiplatelet agent, in a single phase and that exhibit substantial differences in their inherent chemical properties to each other.

Another objective of the present invention is to have a polypill greatly useful in controlling patients exhibiting risk factors for cardiovascular disease by providing the appropriate therapeutic amounts of each of the components of the polypill.

Yet another object of the invention is to facilitate adherence to treatment for the management of patients with cardiovascular disease.

These objectives will be appreciated in detail in the following description.

### BRIEF DESCRIPTION OF THE DRAWINGS

FIG. 1 shows a graph of the evaluation of the dissolution profile of each of the drugs present in the composition of the present invention.

FIG. 2 shows the profile graph in semi-logarithmic scale (semilog) of the mean plasma concentrations of losartan vs. time, obtained from a comparative bioavailability study of a commercial medicament of losartan and the composition of the present invention. Vertical bars indicate the standard error.

FIG. 3 shows the graph in semilog scale of the profile from the average plasma concentrations of the metabolite EXP-3174, with respect to time, obtained from a comparative bioavailability study of a commercial losartan medicament and the composition of the present invention. Vertical bars indicate the standard error.

FIG. 4 shows the profile graph in semilog scale of the average plasma concentrations of pravastatin, vs. time, obtained from a comparative bioavailability study of a commercial pravastatin medicament and the composition of the present invention.

### DETAILED DESCRIPTION OF THE INVENTION

It is well known that in order to control risk factors and prevent cardiovascular events is necessary that the high-risk patients be administered with an effective amount of one or more drugs such as cholesterol lowering agents, antihypertensive agents and/or antiplatelet agents.

It is desirable that these drugs be administered in a single tablet to reduce treatment costs, improve adherence and reduce side effects.

However, until today it has not been possible to develop a stable formulation containing a statin, such as pravastatin, in combination with antihypertensive agents and molecules with acidic properties.

Cholesterol-lowering agents act at different sites and by different mechanisms. For example, in the intestine, cholestyramine combines with bile acids, cholesterol precursors, forming an insoluble complex that is excreted. The mechanism of action of gemfibrozil is not clear, but appears to decrease the hepatic uptake of free fatty acids and decrease peripheral lipolysis. Statins inhibit cholesterol formation by blocking the liver's production of its precursor, as well as by inhibiting the enzyme HMG-CoA reductase required for cholesterol synthesis.

From a pharmacological analysis it was decided that among the cholesterol-lowering agents the use of pravastatin sodium is preferred because it promotes the reduction of incidence of cerebral thrombosis up to a 20%, since by reducing LDL cholesterol levels it stabilizes plaques of atherosclerosis present in the carotid arteries and the aorta and prevents breakage of the same. Also, it has the property to prevent type 2 diabetes mellitus, since the number of incidence of the disease in patients from the group administered with pravastatin was 30% lower than in the group administered with placebo.

On the other hand, it has been found that inhibitors of the renin-angiotensin system, such as angiotensin II antagonists, also called angiotensin II receptor blockers, because they have the ability to partially or completely block angiotensin receptors, namely AT1 receptor, directly causing vasodilation, reduce vasopressin secretion and the production and secretion of aldosterone is reduced, among other actions whose combined effect is blood pressure reduction.

Although it has been shown that ACE inhibitors are effective to modify systemic blood pressure and in selected territories of injury, including glomerular, they also produce negative effects that reduce the adherence of patients, such as cough and angioneurotic edema. Recently it has been shown that angiotensin II receptor blocking has a more direct inhibitory action than the use of ACE inhibitors (hypertension and chronic heart failure) by not interfering with metabolic processes of the renin-angiotensin system.

In recent years extensive evidence has been accumulated which shows the advantages of angiotensin II receptors blockers to reduce urinary albumin excretion in patients suffering from type 2 diabetes, hypertension and microalbuminuria. Two of the studies included patients with established proteinuria and renal failure (RENAAL, IRMA2), the first one was performed with losartan and the second with irbesartan, whereas in the IDNT study, also conducted with irbesartan, patients with normal glomerular filtration were included. The RENAAL (Reduction of Endpoints in NIDDM with the Angiotensin II Receptor Antagonist Losartan) study showed that losartan delays the progression of renal disease in patients with type 2 diabetes mellitus and proteinuria. Therefore, the use of losartan as an antihypertensive agent it is preferred since it has shown good control of high blood pressure and heart failure in patients with high blood pressure and thickening of the left ventricle (left chamber of the heart). Losartan has been shown to decrease the risk of cerebrovascular accident (neurological disorder that results from insufficient blood supply to certain parts of the brain for a period of time). Moreover, in recent years great advances have occurred in the understanding of pathogenesis, prevention and treatment of diabetic nephropathy, especially in two aspects: 1) to elucidate cellular and molecular mechanisms that provoke it; 2) the evidence from recent studies on the effectiveness of glycemic control and antihypertensive treatment to modify the natural history of this complication once that the close association between diabetic nephropathy and the risk of cardiovascular complications has been shown, and in view that they represent the leading cause of morbidity and mortality in these patients.

In the case of antiplatelet agents such as inhibitors of adenosine diphosphate receptors (clopidogrel bisulfate, prasugrel, ticlopidine hydrochloride), adenosine uptake inhibitors (dipyridamole), glycoprotein IIB/IIIA inhibitors (abciximab, eptifibatide, tirofiban, defibrotide), phosphodiesterase inhibitors (cilostazol) and acetylsalicylic acid (aspirin), they are a group of drugs that prevent platelets from agglutinate together and forming a blood clot within a blood vessel which is injured, swollen (inflamed) or that has a buildup of plaque (atherosclerosis) helping to prevent cardiovascular events.

Among the antiplatelet agents acetylsalicylic acid has shown excellent results in inhibiting the formation of thromboxane A2. Aspirin acts by donating an acetylating molecule in the platelet membrane, impeding the formation of thromboxane A2. This action is irreversible and persists throughout the life of the platelet. It also inhibits the formation of prostacyclin (prostaglandin I2) in vascular endothelial cells.

To achieve the formulation of stable compositions of the present invention it was necessary to incorporate protective agents to obtain the combination of two or more drugs with and without coating which is not described in the prior art.

Protective agents for cholesterol-lowering agents can be selected from one or more of the following compounds: calcium carbonate, potassium phosphate, aluminum hydroxide, magnesium hydroxide, magnesium oxide, propyl gallate, ascorbyl palmitate, metabisulfite sodium, butylhydroxyanisole and/or butylhydroxytoluene. A preferred embodiment is the use of mixtures of protective agents in equal amounts to obtain synergistic effects to achieve the stabilization of the composition of the present invention.

Through the development and testing of various formulations, it has been found that there are better results when at least two protective agents are used in a weight ratio of 80-120% compared to the weight of statins, i.e. 25-55 mg of protective agents.

Furthermore, it has been found that in order to obtain a better stability of the composition, firstly the statins can be formulated into granules in combination with the protective agent and other excipients generally used in industry.

The excipients that can be used preferably are the following: diluents such as lactose, microcrystalline cellulose, dextrose, corn starch or calcium phosphate, disintegrating agents such as crospovidone, croscarmellose sodium or sodium starch glycolate; anti-adherents or glidants such as colloidal silicon dioxide, corn starch, and lubricants such as talc, zinc stearate, magnesium stearate, calcium stearate, among other compounds.

In one of the preferred embodiments, the addition of antihypertensive agents into the formulation of statin granules and then mixing with antiplatelet agents in the form of granules or powder using only lubricants as auxiliary agents or excipients is contemplated. The best results are obtained when a mixture of lubricants in a 3:2 ratio is used, wherein the lubricating agent in greater amount is preferably the same lubricant selected for the formulation of statin granules.

Another way to implement the present invention is by formulating antihypertensive agents with antiplatelet agents into granules and then mixing with statins, in the form of powder or granules, with the protective agents and excipients.

In any case, best results are found when the weight ratio of each excipient with respect to final weight of final composition are as follows 50-70% of one or more diluents, 3-6% of one or more disintegrating agents, 0.5-1% of one or more anti-adherents or glidants, and 9-13% of one or more lubricating agents.

Since the recommended amounts of daily intake of each selected drug are widely known, it is preferred that the compositions of the present invention contain a dose of 30-50 mg of pravastatin sodium, 30-60 mg of losartan potassium and 70-100 mg of acetylsalicylic acid. In this case the amount of protective agents to be used is preferably 25-55 mg and the excipients are used preferably in the following proportions, 400-700 mg of diluents, 15-35 mg of disintegrating agents, 2-10 mg of anti-adherents or glidants, and 50-75 mg of lubricating agents.

It is also evident that following the teachings of the present invention the formulation of stabilized statin granules with protective agents to be combined with any other drug with acidic properties can be obtained, wherein its simultaneous administration provides synergistic results, promotes adherence to treatment, or simply reduces associated costs.

The compositions of the present invention can be formulated into tablets which are packed in Aluminum-Aluminum blisters and subjected to stability studies according to ICH international guidelines (International Conference of Harmonization, Guideline for Industry, Stability Testing of New Drug Substances and Products. ICH Q1A (R2) February 2003).

The following illustrates the invention by determining an embodiment of the pharmaceutical composition, as well as an example of the results from stability studies that are consistent.

### Example 1

Various granules-containing compositions were prepared by compaction of 35-40 mg of pravastatin sodium, 50-60 mg of losartan potassium using 35-40 mg of protective agents selected from calcium carbonate, potassium phosphate, aluminum hydroxide, magnesium hydroxide, magnesium oxide, propyl gallate, ascorbyl palmitate, sodium metabisulfite, butylhydroxyanisole and/or butylhydroxytoluene. Additionally the following additives were used: 555 mg microcrystalline cellulose equivalent to 70% by weight of said granules, 24 mg crospovidone equivalent to 3% by weight of said granules, 5 mg colloidal silicon dioxide equivalent to 0.6% by weight of said granules and 66 mg of talc equivalent to 10% by weight of said granules. These granules were mixed with 80-85 mg of acetylsalicylic acid in an 8-9:1 ratio and adding the excipients commonly used in the pharmaceutical industry for tablet manufacturing.

### Example 2

The compositions from Example 1 were subjected to extreme storage conditions.

These conditions were maintained for a month, as follows: temperature 40° C. and 75% relative humidity (RH) and at temperature of 60° C. and 85% RH.

Then, the determination of the contents of each of the drugs was performed. The results are shown in Table 1.

TABLE 1
Determination of drugs content stability

| | | 1 Month | 1 Month |
|---|---|---|---|
| Drug | Initial | 40° C./75% RH | 60° C./85% RH |
| Pravastatin sodium | 99.76% | 98.72% | 98.23% |
| Losartan potassium | 95.88% | 96.31% | 96.64% |
| Acetylsalicylic acid | 94.63% | 96.45% | 96.05% |
| Color of composition | White | White | White |

Considering that the drug content determination may have measurement errors of up to 2%, which is commonly handled in industry, it was noted that the variations are within said range, it was concluded that the composition remains physically and chemically stable. Stability is also corroborated by the fact that no color change was observed.

### Example 3

Assessment of the composition of the present invention formulated into tablets was made, as described in Example 1 to determine their dissolution profiles, according to methods widely known in the industry, in order to evaluate the release of drugs.

The results of these tests are shown in Table 2. According to the results obtained it was observed that all three drugs dissolved properly without exhibiting interference between them.

TABLE 2
Dissolution Profile. Average of % dissolved.

| | Pravastatin | Losartan | Acetylsalicylic |
|---|---|---|---|
| Time | sodium | potassium | acid |
| 10 | 61% | 60% | 12% |
| minutes | | | |
| 20 | 104% | 100% | 34% |
| minutes | | | |
| 30 | 105% | 102% | 56% |
| minutes | | | |
| 40 | 106% | 102% | 73% |
| minutes | | | |
| 50 | 107% | 103% | 84% |
| minutes | | | |
| 60 | 108% | 104% | 88% |
| minutes | | | |

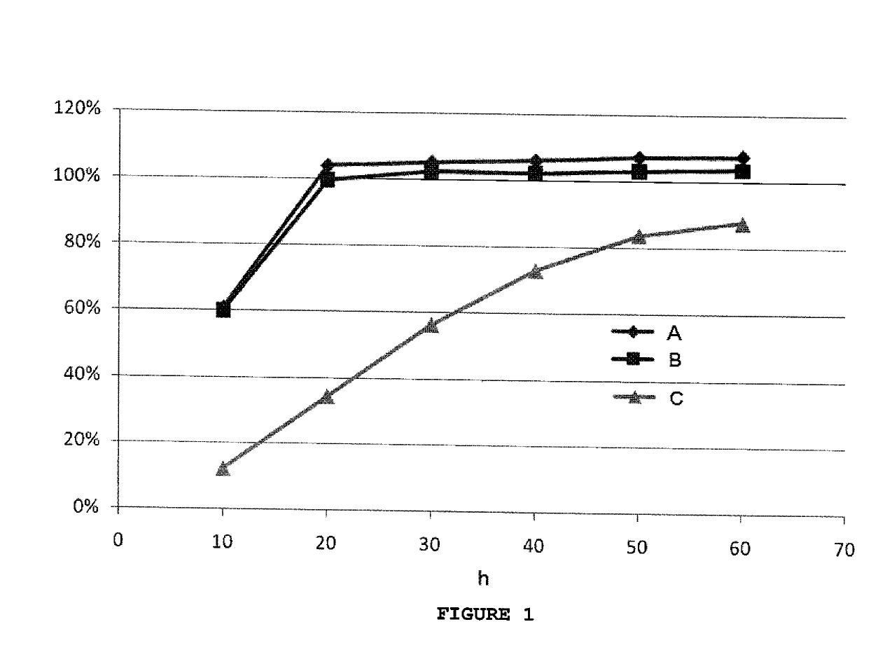

FIG. 1 shows the dissolution profiles, wherein curve A corresponds to pravastatin, curve B to losartan and curve C to acetylsalicylic acid.

### Example 4

Long-term stability studies were conducted on a batch from the composition of the present invention manufactured in accordance with that described in Example 1 but with the addition of coloring agent; these studies were conducted in selected conditions of temperature and humidity, which were 30° C. and 75% RH, and a temperature of 25° C. and 60% RH, showing that the pharmaceutical composition is stable according to the results exemplified in Table 3 up to 9 months, although the study was conducted under the regulatory guidelines for a longer period.

TABLE 3
Stability Test

| | | Time (months) | | | | |
|---|---|---|---|---|---|---|
| Determination | Specifications | 0 | 1 | 3 | 6 | 9 |
| Description | Pink, slotted | No | No | No | No | No |
| | oblong tablet | change | change | change | change | change |
| Content of | 90.0%-110.0% | 98.89 | 98.04 | 95.83 | 98.77 | 99.97 |
| pravastatin | | | | | | |
| sodium | | | | | | |
| Content of | 90.0%-110.0% | 100.32 | 99.24 | 98.50 | 99.85 | 95.33 |
| losartan | | | | | | |
| potassium | | | | | | |
| Content of | 90.0%-110.0% | 98.70 | 101.00 | 98.54 | 98.67 | 95.14 |
| Acetyl- | | | | | | |
| salicylic acid | | | | | | |
| Dissolution of | Q = 80% | 99.79 | 94.77 | 98.09 | 95.97 | 93.37 |
| pravastatin | | | | | | |
| sodium | | | | | | |
| Dissolution of | Q = 80% | 103.01 | 95.28 | 96.38 | 92.77 | 91.66 |
| losartan | | | | | | |
| potassium | | | | | | |
| Dissolution of | Q = 70% | 88.75 | 78.38 | 83.57 | 87.60 | 81.66 |
| Acetyl-salicylic acid | | | | | | |

### Example 5

The evaluation of the bioavailability of the tablets from the composition of the present invention obtained in Example 1 was performed by comparing against commercial products separately containing each of the drugs, according to Williams' 4×4 design with 24 volunteers.

The bioavailability study consisted in administration of each of the following sequences set out in Table 4. Tablets A contain only pravastatin, Tablets B contain only losartan; Tablets C contain only acetylsalicylic acid, and Tablets D correspond to tablets from the composition of the present invention prepared as indicated in Example 1.

TABLE 4

| Sequence | Period 1 | Period 2 | Period 3 | Period 4 |
|---|---|---|---|---|
| 1 | A | B | C | D |
| 2 | B | C | D | A |
| 3 | C | D | A | B |
| 4 | D | A | B | C |

Table 5 shows the average levels of plasmatic concentration per dose, Cp in ng/ml, reached for each drug respect to average time, h in hours.

TABLE 5
Cp/Do values (ng/ml)

| Time | Salicylic | | Metabolite | |
|---|---|---|---|---|
| (h) | acid | Losartan | EXP-3174 | Pravastatin |
| 0.00 | 0 | 0 | 0 | 0 |
| 0.17 | 1.09 | 39.68 | NC | 2.80 |
| 0.33 | 3.97 | 136.75 | NC | 24.93 |
| 0.50 | 8.78 | 190.91 | 11.53 | 72.58 |
| 0.75 | 14.82 | 176.22 | 37.45 | 112.59 |
| 1.00 | 21.07 | 139.85 | 87.26 | 109.75 |
| 2.00 | 28.82 | 113.17 | 183.61 | 76.96 |
| 2.50 | 36.55 | 96.28 | 256.42 | 58.01 |
| 3.00 | 43.28 | 81.49 | 317.82 | 42.46 |
| 4.00 | 49.24 | 57.48 | 377.13 | 25.82 |
| 6.00 | 50.18 | 38.68 | 388.88 | 16.25 |
| 7.00 | 45.43 | 21.82 | 344.19 | 9.73 |
| 8.00 | 25.05 | 12.45 | 224.62 | 13.3 |
| 10.00 | 12.53 | 6.01 | 151.47 | 6.53 |
| 12.00 | 4.21 | 3.06 | 54.14 | 1.71 |
| 24.00 | 1.16 | NC | 12.44 | 1.27 |

NC = not quantifiable

Maximum plasma concentration (Cmax values in ng/mL) achieved for each of the drugs in the tablets of Example 1 of the present invention in each of the volunteers who participated in the bioavailability study was determined, the results are shown in Table 6.

TABLE 6
Cmax values in ng/mL

| | Acetylsalicylic | | Metabolite | |
|---|---|---|---|---|
| Patient | acid | Losartan | EXP-3174 | Pravastatin |
| 1 | 65.29 | 267.9 | 658.03 | 95.48 |
| 2 | 53.65 | 471.24 | 504.95 | 222.5 |
| 3 | 36.74 | 471.92 | 421.81 | 94.57 |
| 4 | 49.75 | 197.35 | 451.81 | 166.05 |
| 5 | 46.29 | 352.07 | 723.94 | 240.56 |
| 6 | 42.13 | 210.81 | 304.89 | 116.98 |
| 7 | 43.38 | 160.26 | 339.25 | 213.94 |
| 8 | 67.05 | 193.97 | 285.83 | 134.12 |
| 9 | 56.64 | 161.16 | 376.89 | 121.44 |
| 10 | 53.85 | 131.46 | 493.71 | 46.13 |
| 11 | 52.82 | 101.9 | 549.29 | 88.9 |
| 12 | 68.95 | 212.91 | 494.04 | 74.64 |
| 13 | 38.83 | 315.44 | 372.7 | 93.94 |
| 14 | 51.07 | 336.58 | 609.66 | 231.41 |
| 15 | 12.97 | 288.57 | 623.97 | 84.4 |
| 16 | 42.37 | 136.6 | 453.48 | 54.86 |
| 17 | 62.59 | 291.12 | 554.44 | 110.42 |
| 18 | 59.8 | 389.17 | 354.73 | 222.88 |
| 19 | 64.73 | 153.4 | 328.97 | 66.03 |
| 20 | 48 | 71.96 | 189.93 | 21.07 |
| 21 | 59.28 | 443.36 | 235.55 | 146.69 |
| 22 | 55.26 | 182.25 | 478.92 | 119.08 |
| 23 | 50.07 | 162.64 | 170.49 | 149.95 |
| 24 | 53.97 | 86.48 | 285.67 | 148.12 |

The comparative evaluation of the various average pharmacokinetic parameters of bioavailability for pravastatin, metabolite EXP-3174 and losartan from drug administration to 24 volunteers involved was performed, according to the sequence of Table 4. The results are shown in Tables 7 to 9.

TABLE 7
Comparative table for pravastatin bioavailability

| Pharmacokinetic | Commercial | Composition | Relative |
|---|---|---|---|
| parameter | product (PC) | (T) | bioavailability |
| Cₘₐₓ (ng/ml) | 123.65 | 127.67 | 103% |
| | (57.09) | (62.17) | |
| Tₘₐₓ (h) | 1.4 | 0.9 | 67% |
| | (1.14) | (0.48) | |
| AUC₀₋ₜ | 361.04 | 345.57 | 96% |
| (h* ng/ml) | (145.01) | (150.18) | |
| AUC_{0-inf} | 368,146 | 354.57 | 96% |
| (h* ng/ml) | (144.75) | (151.85) | |
| λz (h−1) | 0.353 | 0.351 | 99% |
| | (0.163) | (0.134) | |
| T_{1/2} (h) | 2.4 | 2.3 | 94% |
| | (1,174) | (0.874) | |
| Vz/F (ml) | 441,100.37 | 437,560.40 | 99% |
| | (274,765.11) | (233,608.54) | |
| Cl/F (ml/h) | 124,686.68 | 133,662.77 | 107% |
| | (46,694.92) | (57,193.75) | |
| MRT₀₋ₜ (h) | 3.51 | 3.35 | 95% |
| | (0.709) | (0.598) | |
| MRT_{0-inf} (h) | 3.84 | 3.71 | 97% |
| | (0.962) | (0.729) | |

wherein:
Tₘₐₓ=Time to reach Cₘₐₓ
λz=Value of slope of the linear portion of elimination phase
T_{1/2}=λz Half-life
Vz/F=Distribution volume of elimination phase with respect to the fraction of absorbed dose (F)
Cl/F=Total clearance over the fraction of absorbed dose
MRT=Mean residence time.

Regarding the pharmacokinetic parameters of losartan bioavailability, the results are shown below.

TABLE 8
Comparative pharmacokinetic parameters
for losartan bioavailability

| | | | Relative |
|---|---|---|---|
| Pharmacokinetic | Commercial | | bioavailability |
| parameter | product | Composition | (%) |
| Cₘₐₓ (ng/mL) | 262.21 | 241.27 | 92.01 |
| | (185.488) | (120.221) | |
| Tₘₐₓ (hr) | 1.35 | 1.18 | 87.04 |
| | (1.053) | (1.135) | |
| AUC₀₋ₜ | 578.11 | 582.97 | 100.84 |
| (hr* ng/mL) | (197.768) | (170.552) | |
| AUC_{0-inf} | 588.96 | 596.53 | 101.29 |
| (hr* ng/mL) | (198.249) | (167.532) | |
| λ_{z} (hr⁻¹) | 0.51 | 0.51 | 99.32 |
| | (0.135) | (0.168) | |
| T_{1/2} (hr) | 1.43 | 1.48 | 103.51 |
| | (0.330) | (0.418) | |
| Vz/F (mL) | 195,582.66 | 197,024.67 | 100.74 |
| | (89,519.882) | (99,552.178) | |
| Cl/F (mL/hr) | 94,751.85 | 90,602.62 | 95.62 |
| | (33,256.448) | (26,437.618) | |
| MRT₀₋ₜ (hr) | 3.03 | 2.87 | 94.53 |
| | (0.826) | (0.653) | |
| MRT_{0-inf} ₍hr) | 3.21 | 3.10 | 96.55 |
| | (0.820) | (0.849) | |

Finally, the comparative pharmacokinetic parameters for bioavailability of metabolite EXP-3174 were determined.

TABLE 9
Comparative table for bioavailability of metabolite EXP-3174

| | | | Relative |
|---|---|---|---|
| Pharmacokinetic | Commercial | | bioavailability |
| parameter | product | Composition | (%) |
| Cₘₐₓ (ng/mL) | 427.77 | 427.61 | 99.96 |
| | (141.193) | (148.239) | |
| Tₘₐₓ (hr) | 5.7 | 5.5 | 97.43 |
| | (1.494) | (1.571) | |
| AUC₀₋ₜ | 2,982.684 | 3,067.462 | 102.84 |
| (hr* ng/mL) | (911.119) | (1,048.282) | |
| AUC_{0-inf} | 3,035.385 | 3,198.719 | 105.38 |
| (hr* ng/mL) | (903.389) | (1,012.390) | |
| λ_{z} (hr⁻¹) | 0.289 | 0.259 | 89.64 |
| | (0.162) | (0.123) | |
| T_{1/2} (hr) | 2.8 | 3.5 | 121.96 |
| | (0.907) | (2.742) | |
| Vz/F (mL) | 78,087.42 | 86,335.54 | 110.56 |
| | (50,275.537) | (67,387.417) | |
| Cl/F (mL/hr) | 18,395.00 | 17,398.63 | 94.58 |
| | (7,709.838) | (6,413.432) | |
| MRT₀₋ₜ (hr) | 6.89 | 6.85 | 99.46 |
| | (1.363) | (1.721) | |
| MRT_{0-inf} ₍hr) | 7.20 | 7.85 | 109.00 |
| | (1.472) | (4.933) | |

As can be seen, the pharmacokinetic parameters of the stable composition of the present invention are very similar to those shown by the drugs administered separately.

To corroborate this information, profiles of plasma concentrations per dose (Cp/Do) versus elapsed time in hours (h) were constructed in semi-logarithmic scale, with corresponding standard error, from the average results obtained for Pravastatin, Losartan and the metabolite EXP-3174 from the 24 volunteers by the administration of medicaments commercially available (PC) compared with those obtained with the composition of the present invention (T).

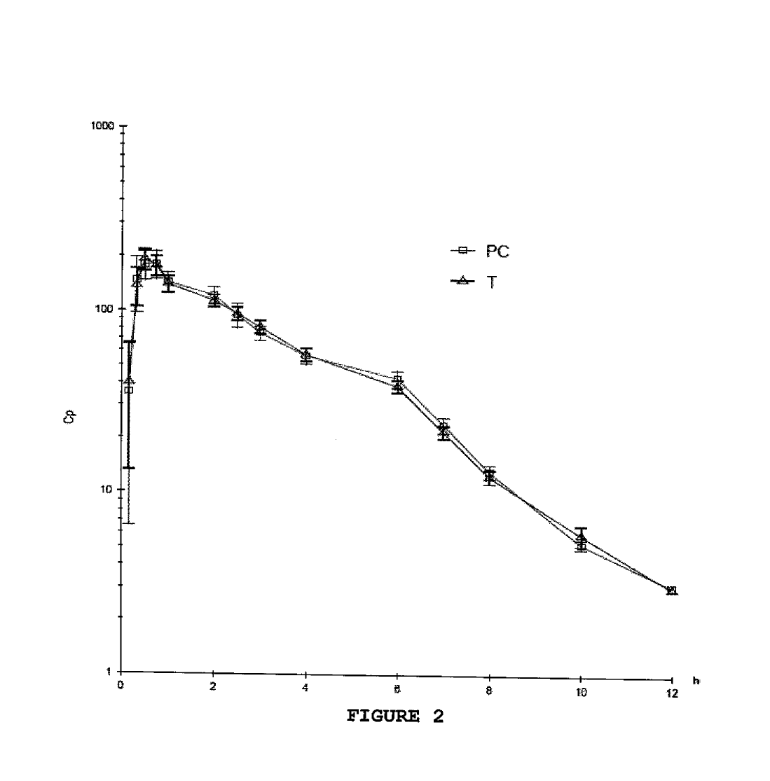

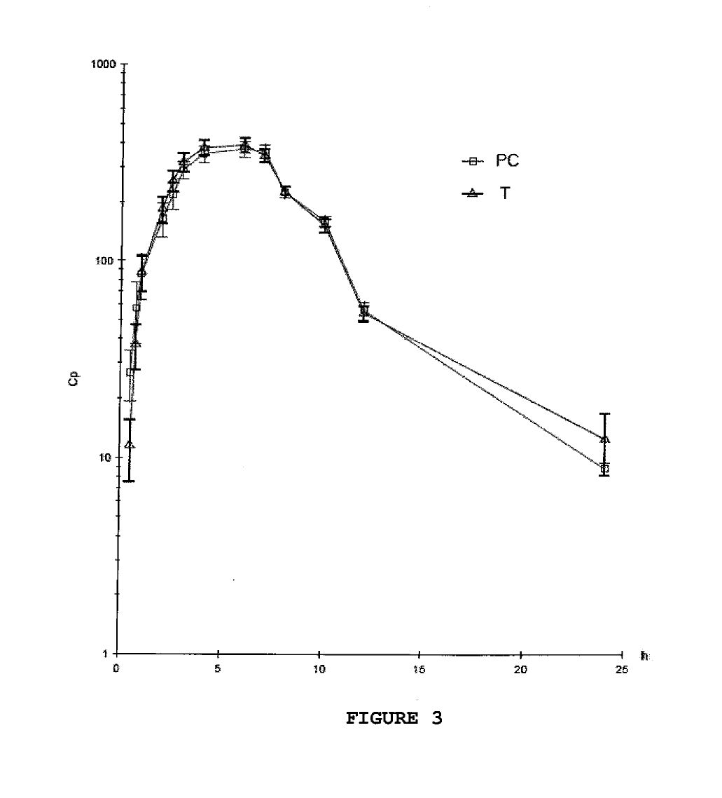

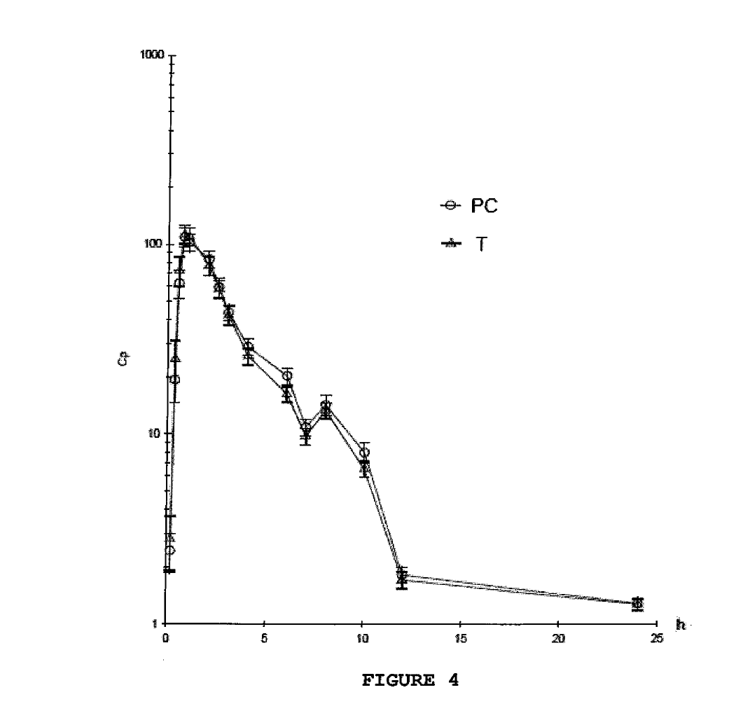

FIG. 2 shows the graph of losartan, FIG. 3 shows the profile of the average plasma concentrations of the metabolite EXP-3174 and FIG. 4 corresponds to Pravastatin.

In these graphs it can be seen that there is a similar bioavailability of the composition of the present invention (T) respect to commercial mono-drug products (PC).

In view of the above mentioned, it is believed that the disclosed invention provides an stable composition of two or more drugs that solves a major technical challenge due to its physicochemical characteristics. Therefore the invention has novelty, inventive step and has industrial applicability, and hence the content of the following claims is claimed as property.

What is claimed is:

1. A stable composition of cholesterol-lowering agents, antihypertensives agents and antiplatelet agents containing granules with 30-50 mg of cholesterol-lowering agents, 30-60 mg of antihypertensive agents and 70-100 mg of antiplatelet agents wherein said composition comprises 400-700 mg of a diluting agent; 30-60 mg of a disintegrating agent, 5-15 mg of an anti-adherent, 90-120 mg of lubricant and 25-55 mg of protective agents.

2. The stable composition according to claim 1, wherein said cholesterol lowering agent is preferably pravastatin sodium.

3. The stable composition according to claim 1, wherein said antihypertensive agent is preferably losartan potassium.

4. The stable composition according to claim 1, wherein said antiplatelet agent is acetylsalicylic acid.

5. The stable composition according to claim 1, wherein said protective agents are selected from the group consisting of: calcium carbonate, aluminum hydroxide, magnesium hydroxide, magnesium oxide, propyl gallate, ascorbyl palmitate, sodium metabisulfite, butylhydroxyanisole, butylhydroxytoluene, and combinations thereof.

6. The stable composition according to claim 1, wherein said cholesterol-lowering agents, antihypertensives agents, antiplatelet agents, diluting agents, disintegrating agents, anti-adherents, lubricants and protective agents are present in a single phase.
